(19) United States
(12) Patent Application Publication
(10) Pub. No.: US 2003/0017152 A1
(43) Pub. Date: Jan. 23, 2003

(54) Non-lethal methods for conditioning a recipient for bone marrow transplantation

(76) Inventor: Suzanne T. Ildstad, Prospect, KY (US)

Correspondence Address: HOGAN & HARTSON LLP, ONE TABOR CENTER, SUITE 1500, 1200 SEVENTEENTH ST, DENVER, CO, 80202, US

(21) Appl. No.: 10/134,016

(22) Filed: Apr. 26, 2002

Related U.S. Application Data
(63) Continuation-in-part of application No. PCT/US01/46126, filed on Nov. 14, 2001.
(60) Provisional application No. 60/249,048, filed on Nov. 14, 2000.

(51) Int. Cl. A61K 48/00; A61K 39/395

(52) U.S. Cl. 424/144.1; 424/93.21; 424/93.7; 514/44; 514/110

(57) ABSTRACT

Hematopoietic chimerism induces donor-specific tolerance to solid organ grafts. The clinical application of this technique is limited by the morbidity and mortality of conventional bone marrow transplantation (BMT). Conditioning for engraftment is nonspecific, utilizing myeloablation plus nonspecific immunosuppression. In the present study we have characterized which cells in the recipient hematopoietic microenvironment prevent allogeneic marrow engraftment. Mice defective in production of αβ-TCR cells, γδ-TCR cells; αβ- plus γδ-TCR cells; CD8 cells and CD4 cells were transplanted with MHC-disparate allogeneic bone marrow. In normal mice, 500 cGy total body irradiation (TBI) plus cyclophosphamide (200 mg/kg) on day +2 is required for engraftment of allogeneic hematopoietic stem cells (HSC). Mice lacking both αβ- and γδ-TCR⁺ cells engrafted when conditioned with 0 to 300 cGy TBI alone, suggesting that αβ plus γδ T cells in the host play a critical and non-redundant role in preventing engraftment of allogeneic bone marrow. When mice were conditioned with 300 cGy TBI plus a single dose of cyclophosphamide on day +2, all mice engrafted except for mice defective in production of CD4⁺ cells. Moreover, CD8 KO mice engrafted without TBI if administered cyclophosphamide on day +2 relative to the marrow infusion. These results suggest that different cell populations in host marrow with different mechanisms of action play a role in the resistance to engraftment of allogeneic bone marrow. Both αβ-TCR⁺ and γδ-TCR⁺ T-cells play an important and non-redundant role in the marrow rejection response. In addition, the CD8⁺ cell effector function is mechanistically different from that for conventional T-cells and independent of CD4⁺ T-helper cells. Targeting of specific recipient cellular populations may permit conditioning approaches to allow mixed chimerism with minimal morbidity.

### CROSS-REFERENCE TO OTHER APPLICATIONS

This patent application is a continuation-in-part of PCT/USO1/46126, filed Nov. 14, 2001 and entitled “Non-Lethal Methods For Conditioning A Recipient For Bone Marrow Transplantation” and this patent application claims benefit of Provisional Application No. 60/249,048 filed Nov. 14, 2000 and entitled “Non-Lethal Methods For Conditioning A Recipient For Bone Marrow Transplantation”. All of the above-referenced applications are incorporated herein by reference.

### CONTRACTUAL ORIGIN OF THE INVENTION

This research was supported in part by the National Institutes of Health, grant DK43901-07. The government has certain rights in the invention.

### BACKGROUND OF THE INVENTION

1. Field of the Invention

The present invention relates to non-lethal methods of conditioning a recipient for bone marrow transplantation. In particular, the present invention relates to cell specific conditioning strategies which result in the depletion and more preferably the elimination of αβ-, γδ-TCR⁺ T cells and/or CD8⁺ T-cells in the recipient hematopoietic microenvironment through the use of antisense DNA technology, cell type-specific antibodies and/or cell type-specific cytotoxic drugs.

2. Description of the State of Art

The transfer of living cells, tissues, or organs from a donor to a recipient, with the intention of maintaining the functional integrity of the transplanted material in the recipient defines transplantation. Transplants are categorized by site and genetic relationship between the donor and recipient. An autograft is the transfer of one's own tissue from one location to another; a syngeneic graft (isograft) is a graft between identical twins; an allogeneic graft (homograft) is a graft between genetically dissimilar members of the same species; and a xenogeneic graft (heterograft) is a transplant between members of different species.

A major goal in solid organ transplantation is the permanent engraftment of the donor organ without a graft rejection immune response generated by the recipient, while preserving the immunocompetence of the recipient against other foreign antigens. Typically, in order to prevent host rejection responses, nonspecific immunosuppressive agents such as cyclosporine, methotrexate, steroids and FK506 are used. These agents must be administered on a daily basis and if stopped, graft rejection usually results. However, a major problem in using nonspecific immunosuppressive agents is that they function by suppressing all aspects of the immune response, thereby greatly increasing a recipient's susceptibility to opportunistic infections, rate of malignancy, and end-organ toxicity. The side effects associated with the use of these drugs include opportunistic infection, an increased rate of malignancy, and end-organ toxicity (Dunn, D. L., Crit. Care. Clin., 6:955 (1990)). Although immunosuppression prevents acute rejection, chronic rejection remains the primary cause of late graft loss (Nagano, H., et al, Am J. Med. Sci, 313:305-309 (1997)).

For every organ, there is a fixed rate of graft loss per annum. The five-year graft survival for kidney transplants is 74% (Terasaki, P. I., et al., UCLA Tissue Typing Laboratory (1992)). Only 69% of pancreatic grafts, 68% of cardiac transplants and 43% of pulmonary transplants function 5 years after transplantation (Opelz, G., Transplant Proc, 31:31S-33S (1999)).

The only known clinical condition in which complete systemic donor-specific transplantation tolerance occurs is when chimerism is created through bone marrow transplantation. (See, Qin, et al, J. Exp. Med., 169:779 (1989); Sykes, et al., Immunol. Today, 9:23-27 (1988); and Sharabi, et al., J. Exp. Med., 169:493-502 (1989)). This has been achieved in neonatal and adult animal models as well as in humans by total lymphoid or body irradiation of a recipient followed by bone marrow transplantation with donor cells. The success rate of allogeneic bone marrow transplantation is, in large part, dependent on the ability to closely match the major histocompatability complex (MHC) of the donor cells with that of the recipient cells to minimize the antigenic differences between the donor and the recipient, thereby reducing the frequency of host-versus-graft responses and graft-versus-host disease (GVHD). In fact, MHC matching is essential, only a one or two antigen mismatch is acceptable because GVHD is very severe in cases of greater disparities.

The major histocompatability complex (MHC) is a cluster of closely linked genetic loci encoding three different classes (class I, class II, and class III) of glycoproteins expressed on the surface of both donor and host cells that are the major targets of transplantation rejection immune responses. The MHC is divided into a series of regions or subregions and each region contains multiple loci. An MHC is present in all vertebrates, and the mouse MHC (commonly referred to as H-2 complex) and the human MHC (commonly referred to as the Human Leukocyte Antigen or HLA) are the best characterized.

The role of MHC was first identified for its effects on tumor or skin transplantation and immune responsiveness. Different loci of the MHC encode two general types of antigens which are class I and class II antigens. In the mouse, the MHC consists of 8 genetic loci: Class I is comprised of K and D, class II is comprised of I-A and /or I-E. The class II molecules are each heterodimers, comprised of I-Aα and I-Aβ and/or I-Eα and I-Eβ. The major function of the MHC molecule is immune recognition by the binding of peptides and the interaction with T cells, usually via the αβ T-cell receptor. It was shown that the MHC molecules influence graft rejection mediated by T cells (Curr. Opin. Immunol., 3:715 (1991), as well as by NK cells (Annu. Rev. Immunol., 10:189 (1992); J. Exp. Med., 168:1469 (1988); Science, 246:666 (1989). The induction of donor-specific tolerance by HSC chimerism overcomes the requirement for chronic immunosuppression. See, Ildstad, S. T., et al., Nature, 307:168-170 (1984), Sykes, M., et al., Immunology Today, 9:23-27 (1998), Spitzer, T. R., et al., Transplantation, 68:480-484 (1999)). Moreover, bone marrow chimerism also prevents chronic rejection (Colson, Y., et al., Transplantation, 60:971-980 (1995); and Gammie, J. S., et al., In Press Circulation (1998)). The association between chimerism and tolerance has been demonstrated in numerous animal models including rodents, (Ildstad, S. T., et al., Nature, 307:168-170 (1984); and Billingham, R. E., et al., Nature, 172:606 (1953)) large animals, primates and humans (Knobler, H. Y., et al., Transplantation, 40:223-225 (1985); Sayegh, M. H., et al., Annals of Internal Medicine, 114:954-955 (1991)).

The cells of all hematopoietic lineages are produced by hematopoietic stem cells (HSC). During this procedure, some HSC retain a long-term multilineage repopulating potential (self-renewal); and some HSC may only retain a short-term multilineage repopulating potential and differentiate to produce progeny. The major purified HSC transplantation-related complications include graft rejection and graft failure. The outcome for engraftment of highly purified HSC in the major histocompatability complex (MHC)-matched recipients is different from that for MHC-disparate allogeneic recipients (El-Badri, N. S., Good, R. A., (1993); and Kaufman, C. L., S. Cell Biochem Suppl., 18:A112 (1994)).

The hematopoietic microenvironment plays a major role in the engraftment of HSC. In addition to being a source of growth factors and cellular interactions for the survival and renewal of stem cells, it may also provide physical space for these cells to reside. A number of cell types collectively referred to as stromal cells are found in the vicinity of the hematopoietic stem cells in the bone marrow microenvironment. These cells include both bone marrow-derived CD45⁺ cells and non-bone marrow-derived CD45^{- cells, such as adventitial cells, reticular cells, endothelial cells and adipocytes.}

Recently, Ildstad, et al., identified another bone marrow-derived cell type known as hematopoietic facilitatory cells, which when co-administered with donor bone marrow cells enhance the ability of the donor cells to stably engraft in allogeneic and xenogeneic recipients. See, U.S. Pat. No. 5,772,994 which is incorporated herein by reference. The facilitatory cells and the stromal cells occupy a substantial amount of space in a recipient's bone marrow microenvironment, which may present a barrier to donor cell engraftment. Hematopoietic stem cells bind to facilitatory cells in vitro and in vivo. Thus, the facilitatory cells may provide physical space or niche on which the stem cells survive and are nurtured. It is therefore believed to be desirable to develop conditioning regimens to specifically target and eliminate these and other stromal cell populations in order to provide the space necessary for the hematopoietic stem cells and the associated facilitatory cells in a donor cell preparation to engraft without the use of lethal irradiation. See, U.S. Pat. Nos. 5,635,156 and 5,876,692 which are also incorporated herein by reference.

Until fairly recently, it was believed by those skilled in the art that lethal conditioning of a human recipient was required to achieve successful engraftment of donor bone marrow cells in the recipient. Now, a number of sublethal conditioning approaches in an attempt to achieve engraftment of allogeneic bone marrow stem cells with less aggressive cytoreduction have been reported in rodent models (Mayumi and Good, J. Exp. Med., 169:213 (1989); Slavin, et al., J. Exp. Med., 147(3):700 (1978); McCarthy, et al, Transplantation, 40(1):12 (1985); Sharabi, et al., J. Exp. Med., 172(1):195 (1990); and Monaco et al., Ann. NY Acad. Sci, 129:190 (1966)). However, reliable and stable donor cell engraftment as evidence of multilineage chimerism was not demonstrated, and long-term tolerance has remained a question in many of these models (Sharabi and Sachs, J. Exp. Med., 169:493 (1989); Cobbold, et al., Immunol Rev., 129:165 (1992); and Qin, et al, Eur. J. Immunol., 20:2737 (1990)). Moreover, reproducible engraftment has not been achieved, especially when multimajor and multiminor antigenic disparities existed. The requirement for lethal or sublethal irradiation of the host which renders it totally or partially, respectively, immunocompetent however, poses a significant limitation to the potential clinical application of bone marrow transplantation to a variety of disease conditions, including solid organ or cellular transplantation, sickle cell anemia, thalassemia and aplastic anemia.

Early work by Wood and Monaco attempted to induce tolerance using bone marrow plus anti-lymphocyte serum (ALS) in partial MHC-matched donor-recipient combinations (Wood, et al, Trans. Proc., 3(1):676 (1971); Wood and Monaco, Transplantation, (Baltimore) 23:78 (1977). Even in this semi-allogeneic system, F₁ splenocytes were required to facilitate the induction of tolerance, and thymectomy was required for stable long-term tolerance. The additional requirement for splenocytes and thymectomy made potential clinical applicability of such an approach unlikely. However, these studies identified two key factors required for induction of tolerance: an antigenic source of tolerogen, which is not only involved in tolerance induction, but must also be present at least periodically for permanent antigen-specific tolerance, and a method to tolerize or prevent activation of new T cells from the thymus, i.e. thymectomy, or intrathymic clonal deletion.

Attempts to induce tolerance to allogeneic bone marrow donor cells using combinations of depleting and non-depleting anti-CD4 and CD8 monoclonal antibodies (mAb) resulted in only transient tolerance to MHC-compatible combinations (Cobbold, et al., Immunol Rev, 129:165 (1992); and Qin, et al., Eur. J. Inmunol., 20:2737 (1990). 6Gy of TBI was required to obtain stable engraftment and tolerance when MHC-disparate bone marrow was utilized (Cobbold, et al., Transplantation, 42:239 (1986). Sharabi and Sachs attributed the failure of anti-CD4/CD8 mAb therapy alone to the inability of mAb to deplete T cells from the thymus, since persistent cells coated with mAb could be identified in this location (Sharabi and Sachs, J. Exp. Med., 169:493-502 (1989). However, subsequent attempts to induce tolerance by the addition of 7Gy of selective thymic irradiation prior to donor bone marrow transplantation also failed. Engraftment was only achieved with the addition of 3Gy of recipient TBI.

The risk inherent in tolerance-inducing conditioning approaches must be low when less toxic means of treating rejection are available or in cases of morbid, but relatively benign conditions. In addition to solid organ transplantation, hematologic disorders, including aplastic anemia, severe combined immunodeficiency (SCID) states, thalassemia, diabetes and other autoimmune disease states, sickle cell anemia, and some enzyme deficiency states, may all significantly benefit from a nonlethal preparative regimen which would allow partial engraftment of allogeneic or even xenogeneic bone marrow to create a mixed host/donor chimeric state with preservation of immunocompetence and resistance to GVHD. For example, it is known that only approximately 40% of normal erythrocytes are required to prevent an acute sickle cell crisis (Jandle, et al., Blood, 18(2) (1961); Cohen, et al., Blood, 18(2):133 (1961); and Cohen, et al., Blood, 76(7) (1984)), making sickle cell disease a prime candidate for an approach to achieve mixed multilineage chimerism. Although the morbidity and mortality associated with the conventional full cytoreduction currently utilized for allogeneic bone marrow transplant cannot be justified for relatively benign disorders, the induction of multilineage chimerism by a less aggressive regimen certainly remains a viable option.

Permanent tolerance to donor antigens has been documented in H-2 (MHC) identical or congenic strains with minimal therapy and/or transplantation of donor skin drafts or splenocytes alone (Quin, et al, Eur. J. Immunol., 20:2737 (1990). However, similar attempts to achieve engraftment and tolerance in MHC-mismatched combinations have not enjoyed the same success. In most models, only transient donor-specific tolerance has been achieved (Mayumi, et al., Transplantation, 44(2):286 (1987); Mayumi, et al., Transplantation, 42(4):286 (1986); Cobbold, et al., Eur. J. Immunol., 20:2747 (1990); and Cobbold, et al., Seminars in Immunology, 2:377 (1990)).

When greater genetic disparities exist between donor and recipient, more conditioning is required to achieve engraftment. Engraftment across MHC barriers has been achieved with low dose irradiation in combination with pre-treatment of the host with depleting and nondepleting CD4 and CD8 mAbs, (Cobbold, S. P., et al., Nature, 328:164-166 (1986)), or the use of mAbs in combination with thymic irradiation (Sharabi, Y., et al., Journal of Experimental Medicine, 169:493-502 (1989)). In MHC plus minor antigen disparate mice conditioned with 1 mg ALG on day −3 and 200 mg/kg cyclophosphamide on day +2 and transplanted with 15×10⁶ allogeneic bone marrow cells, durable multi-lineage chimerism occurs with as low as 200 cGy total body irradiation (Colson, Y. L., et al., Journal of Immunology, 157:2820-2829 (1996)). When 30×10⁶ bone marrow cells are transplanted, engraftment can be achieved with 100 cGy TBI in this model (Colson, Y. L., Journal of Immunology; 157:2820-2829 (1996)). Replacement of ALG with in vivo administration of anti-CD4 and anti-CD8 antibodies results in engraftment in 100% of recipients, and the level of chimerism is actually higher (Exner, B. G., et al., Surgery, 122:211-227 (1997)). Moreover, anti-CD8 mAb alone is more efficient at ensuring engraftment than ALG (Exner, B. G., et al., Surgery, 122:211-227 (1997)). The caveat to these studies is that mAb conditioning of the recipient in vivo may not fully remove the effector cell population targeted.

The administration of a combination of mAb plus chemotherapeutic agents such as cyclophosphamide has also been successful in achieving engraftment in closely matched donor and recipient combinations. Mayumi, H., et al., Transplantation Proceedings, 20:139-141 (1998).

T-cells have been implicated as the primary effector cells in solid organ allograft rejection. Eto, et al., described that targeting αβ-TCR⁺ T-cells significantly prolonged survival of skin grafts. While the same effect could be achieved by targeting CD3⁺ T-cells, animals prepared by depletion of αβ-TCR⁺ cells demonstrated relatively superior immunocompetence (Eto, M., et al., et al., Immunology, 81:198-204 (1994)). A critical and non-redundant role for host αβ-TCR⁺ T-cells as effector cells in the rejection of heart allografts, since TCR-β KO mice did not reject MHC-disparate cardiac allografts was recently reported (Exner, B. G., et al., Surgery, 126:121-126 (1999)). Bone marrow transplantation (BMT) from normal B10.BR donors restored the immunocompetence to reject third-patty cardiac allografts in TCR-β KO mice (Exner, B. G., et al., Surgery, 126:121-126 (1999)).

T-cells also play an important role in the rejection of bone marrow grafts. When Kernan, et al., characterized the cells present in recipients of HLA-mismatched bone marrow grafts at the time of rejection, they found that graft failure was associated with the emergence of donor-reactive T-cells. (Keman, N. A., et al., Transplantation, 43:842-847 (1987)). Other groups describe that CD2⁺, CD3⁺ and CD8⁺ T-cells of recipient origin in the peripheral blood of bone marrow recipients effectively inhibit the proliferation and differentiation of donor bone marrow cells in vitro (Bierer, B. E., et al., Transplantation, 46:835-839 (1988)).

As discussed in detail above, conditioning of the recipient with a combination of cytoreductive plus immunosuppressive agents is required to achieve engraftment of MHC-disparate marrow (Colson, Y. L., et al., Journal of Immunology, 157:2820-2829 (1997); Gamnu, J. S., et al, Experimental Hematology, 26:927-935 (1998)). For the most part, the approach to cytoreduction has involved nonspecific immunosuppressive agents, such as irradiation and busulfan, which have a broad specificity and poorly defined mechanism of action. If those specific host components which regulate engraftment could be defined, more specific approaches to target only those specific host cells responsible for alloresistance to engraftment would be possible.

Therefore, there remains a need for non-lethal methods of conditioning a recipient for allogeneic bone marrow transplantation that would result in stable mixed multilineage allogeneic chimerism and long term-donor-specific tolerance.

### SUMMARY OF THE INVENTION

Accordingly, it is an object of this invention to provide methods for conditioning a recipient for bone marrow transplantation which eliminates the need for nonspecific immunosuppressive agents, lethal irradiation and/or other myeloablative agents, such as but not limited to busulfan.

It is another object of the present invention to identify which cells in the host recipient microenvironment influence alloresistance to engraftment.

It is yet another object of the invention to deplete or preferably eliminate those cells in the host environment which influence alloresistance to engraftment thereby conditioning the recipient for engraftment.

It is a further object of the present invention to provide methods for treating a variety of diseases and disorders with minimal morbidity.

Additional objects, advantages, and novel features of this invention shall be set forth in part in the description and examples that follow, and in part will become apparent to those skilled in the art upon examination of the following or may be learned by the practice of the invention. The objects and the advantages of the invention may be realized and attained by means of the instrumentalities and in combinations particularly pointed out in the appended claims.

To achieve the foregoing and other objects and in accordance with the purposes of the present invention, as embodied, the method of this invention comprises depleting and preferably eliminating from the recipient hematopoietic environment αβ-TCR⁺ cells, γδ-TCR⁺ cells, and/or CD8⁺ cells.

### BRIEF DESCRIPTION OF THE DRAWINGS

The accompanying drawings, which are incorporated in and form a part of the specifications, illustrate the preferred embodiments of the present invention, and together with the description serve to explain the principles of the invention.

In the Drawings

FIGS. 1-4 graphically illustrate the influence of TBI dose on engraftment and the level of chimerism in TCR-β/δ double KO mice. TCR-β/δ KO mice were transplanted with 15×10⁶ bone marrow cells from B10.BR donors after conditioning with TBI doses ranging from 0-300 cGy on day 0. Animals were typed by flow cytometric analysis monthly for up to 5 months after BMT. n =number of animals in each experiment. FIG. 1 demonstrates the percentage of animals with engraftment at 1 month post BMT; FIG. 2 demonstrates the level of donor chimerism at 1 month post BMT; FIG. 3 demonstrates the level of donor chimerism at 3 months post BMT; and FIG. 4 demonstrates the level of donor chimerism at 5 month post BMT.

FIGS. 5-7 graphically illustrate the characteristics of engraftment in KO mice conditioned with TBI plus CyP. B6 control mice and mice deficient in the production of γδ-TCR⁺ cells (TCR-δKO), αβ-TCR⁺ cells (TRC-β KO), both αβ- and γδ-TCR⁺ cells (TCR-β/δ double KO), CD4 (CD4 KO) or CD8 (CD8 KO) were conditioned with 300 cGy TBI, transplanted with 15×10⁶ bone marrow cells from B10.BR donors and given 200 mg/kg cyclophosphamide (CyP) (i.p.) on day +2. FIG. 5 represents the frequency of engraftment. FIG. 6 demonstrates the level of chimerism in animals that engrafted percentage of donor cells in PBL) 1 month as assessed by PBL typing. FIG. 7 demonstrates the level of chimerism in animals that engrafted (percentage of donor cells in PBL) more than 3 months after as assessed by PBL typing.

FIG. 8 graphically illustrates the characteristics of engraftment in KO mice conditioned with TBI alone. To evaluate which cell subsets were sensitive to cyclophosphamide (CyP) in normal recipients, CD8 KO, TCR-β KO, TCR-δ KO, and TCR-β/δ KO mice were conditioned with 300 cGy TBI alone. Recipients were transplanted with 15×10⁶ bone marrow cells from B10.BR donors on the same day with TBI. The figure shows the percentage of animals that engrafted 1 month post-BMT.

FIGS. 9-11 graphically illustrate the TBI Dose-titration in CD8 KO mice conditioned with post-transplant cyclophosphamide (CyP). FIG. 9 graphically represents CD8 KO mice that were conditioned with TBI doses ranging from 0-300 cGy on day 0 and transplanted with 15×10⁶ allogeneic B10.BR bone marrow cells 4 to 6 hours after. Two hundred mg/kg CyP was administered 2 days after BMT. FIG. 10 demonstrates the level of chimerism after 1 month following BMT; and FIG. 11 demonstrates the level of chimerism after 6 months following BMT.

FIG. 12 demonstrates the detection of multilineage chimerism in representative mixed allogeneic chimera in CD8-KO mice by 3 color flow cytometry. Recipient CD8 KO mice were conditioned with TBI followed by 200 mg/kg cyclophosphamide 2 days after transplantation with 15×10⁶ untreated bone marrow cells. Multilineage typing was performed by using peripheral blood 4 months after reconstitution (n=4). All 4 animals showed multi-lineage chimerism. B-cells (B220⁺ cells), T-cells (CD4⁺, αβ-TCR⁺ and γδ-TCR⁺ cells), natural killer cells (NK1.1⁺ cells), macrophages (MAC-1⁺ cells), and granulocytes (GR1⁺ cells) of recipient and donor type were present in all animals.

FIG. 13 three-color flow cytometric analysis of CD8⁺ cells in chimeric CD8-KO mice. Recipient CD8 KO mice were conditioned with TBI followed by 200 mg/kg cyclophosphamide 2 days after transplantation with 15×10⁶ untreated bone marrow cells. Naïve recipient CD8 KO mice, shown in box A and donor B10.BR mice, shown in box B were used as controls. The CD8 lineage deficient in the CD8 knock-out mice was found in the transplant recipients and was of donor origin, as demonstrated in box C. There were no recipient-type CD8⁺ cells detected.

FIG. 14 is a graphic representation of NK subset expression in bone marrow and spleen of KO mice. NK subset expression was enumerated for bone marrow and spleen from TCR-β KO, TCR-δ, TCR-β/δ KO, CD8 KO mice and the B6 control mice using 4 color cytometry. NK1 .1 ( [symbol] ) , 5E6 ( [symbol] ), T/NK (NK1.1 and TCR positive) ( [symbol] ), CD8/NK1.1 ( [symbol] ) and 2B4 ( [symbol] ). Each bar represents the mean of 3 mice and their standard deviations.

FIG. 15 is a graphic representation of the kinetics of chimerism on subjects without donor T cells.

FIG. 16 is a graphic representation of the kinetics of chimerism on subjects with donor T cells.

FIG. 17 is a graphic representation of donor-specific tolerance to skin grafts in mixed chimerisms.

### DETAILED DESCRIPTION OF THE PREFERRED EMBODIMENT

The present invention is founded on the discovery that host αβ-TCR⁺ and γδ-TCR⁺ T-cells play a critical and nonredundant role in the resistance to engraftment of allogeneic bone marrow. As discussed in further detail below, animals deficient in the production of αβ- and γδ-TCR⁺ T-cells are significantly enhanced in their ability to accept allogeneic bone marrow grafts compared to immunocompetent controls. Mice deficient in production of αβ-TCR⁺ cells alone exhibit similar enhanced engraftment only if cyclophosphamide is administered two days after bone marrow transplant (BMT) after conditioning with 300 cGy total body irradiation (TBI). Mice lacking of production of γδ-TCR⁺ T-cells exhibit enhanced engraftment, although to a lesser extent than αβ-TCR⁺ cells, demonstrating that these cells in the host also play a role in resistance to allogeneic bone marrow engraftment. This finding is supported by the fact that only mice deficient in production of αβ and γδ cells (TCR-β/δ KO) reliably engraft with low TBI dose alone or even no TBI without requiring cyclophosphamide, confirming that both αβ- and γδ-TCR⁺ cells in the host function in a nonredundant and critical fashion in alloresistance to engagement. These data therefore implicate αβ plus γδ T-cells rather than NK cells as the primary effector cells in marrow graft rejection in allotransplantation.

Thus, the present invention relates to non-lethal methods of conditioning a recipient, which may be any mammal and preferably human, for bone marrow transplantation. These methods include the use of anti-sense DNA technology, non-lethal doses of irradiation, cell type-specific antibodies, cell-type specific cytotoxic drugs or a combination thereof. n particular, the present invention encompasses an approach to make space in a recipient's bone marrow by targeting only critical cell populations in the hematopoietic microenvironment.

The invention is discussed in more detail in the subsections below, solely for the purpose of description and not by way of limitation. For clarity of discussion, the specific procedures and methods described herein are exemplified using a murine model; they are merely illustrative for the practice of the invention. Analogous procedures and techniques are equally applicable to all mammalian species, including human subjects.

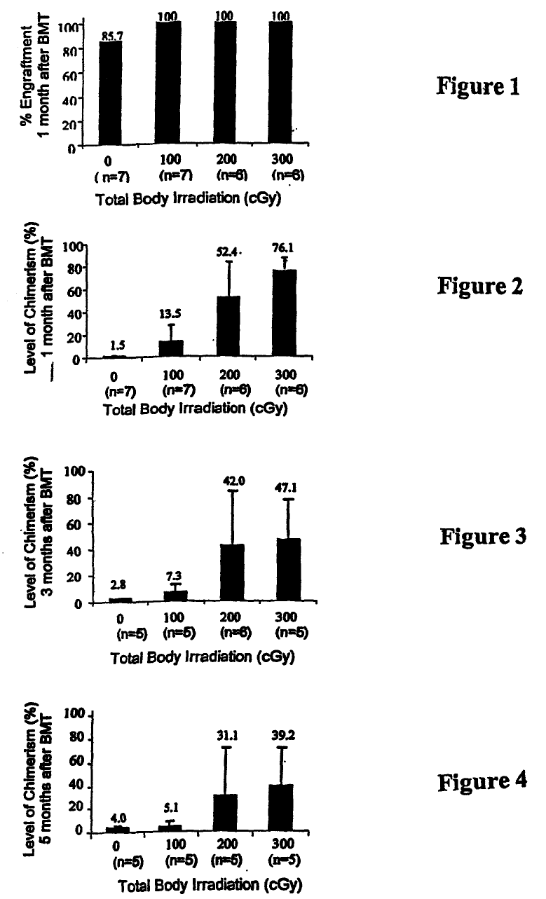

The present invention culminates from the initial evaluation and identification of which specific cell populations in the host hematopoietic microenvironment are the gatekeepers for engraftment of allogeneic marrow using knockout mice (KO). In these animals (KO mice) the gene for the expression of certain cell surface molecules is disrupted so that they cannot produce these cells. Thus, no residual cells are present in these animals. Typically, a minimum of 700 cGy of TBI is required for conditioning in normal mice. In striking contrast, however according to the present invention, durable multi-lineage engraftment of allogeneic marrow was achieved with only 300 cGy TBI in mice lacking both αβ and γδ cells (TCR-β/δ KO) suggesting that host αβ-TCR and γδ-TCR cells play a critical role in allorejection. In order to characterize the minimum effective TBI dose that allows allogeneic engraftment in TCR-βδ KO mice, recipients (H-2^{b}, that are deficient in producing functional αβ- and γδ-TCR T-cells) were conditioned with 0 to 300 cGy TBI and transplanted with 15×10⁶ B10.BR (H-2^{k}, having genes for the production of β-chain and δ-chain of TCR disrupted) bone marrow cells, as shown in FIG. 1. Chimerism was assessed by flow cytometric analysis and the results are shown in FIGS. 2-4. 100% of mice conditioned with 300, 200 or 100 cGy TBI engrafted and the levels of donor chimerism were 76.1±10.4%, 52.4±30.4% and 13.5±14.3%, respectively (FIG. 2). 85.7% of TCR β/δ double KO mice engrafted without any TBI conditioning with 1.5±0.51% of donor chimerism on 28 days. The engraftment was durable as assessed monthly for up to 6 months. The level of chimerism for all groups was directly correlated with the degree of conditioning.

Donor-type skin grafts were accepted by chimeras, while the third-party NOD (H2K^{d}) skin grafts were rejected promptly. The results of this study suggest that durable chimerism and donor-specific tolerance could be achieved in mice deficient in producing functional αβ-TCR and γδ-TCR cells even without any conditioning. Targeting αβ-TCR⁺ and γδ-TCR⁺ in the recipient hematopoietic environment could provide a valuable strategy in the development of clinical protocols for induction of mixed allogeneic chimerism resulting in donor-specific tolerance with minimum morbidity.

Methods for targeting αβ-TCR⁺ and γδ-TCR⁺ in the recipient hematopoietic environment is discussed in further detail below and therefore the present invention encompasses and contemplates the use of antibodies, antisense DNA technology and non-lethal doses of irradiation as ways of depleting and preferably eliminating αβ-TCR⁺ and γδ-TCR⁺ cells in the recipient hematopoietic environment.

Various procedures known in the art may be used for the production of polyclonal antibodies to antigens of cells making up the hematopoietic microenvironment of the host, including but is not limited to αβ-TCR⁺, γδ-TCR⁺, and/or CD8⁺ cells. For the production of antibodies, various host animals can be immunized by injection with purified or partially purified hematopoietic cells such as stromal cells including but not limited to rabbits, hamsters, mice, rats, etc. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, Ricin and potentially useful human adjuvants such as BCG (bacille Calmette-Guerin) and Corynebacterium parvum.

A monoclonal antibody to antigens of αβ-TCR⁺, γδ-TCR⁺, and/or CD8⁺ cells may be prepared by using any technique which provides for the production of antibody molecules by continuous cell lines in culture. These include but are not limited to the hybridoma technique originally described by Kohler and Milstein, Nature, 256: 495-497 (1975), and the more recent human B-cell hybridoma technique (Kosbor, et al., Immunology Today, 4:72 (1983); Cote, et al., Proc. Natl. Acad. Sci., USA, 80:2026-2030 (1983) and the EBV-hybridoma technique (Cole, et al., Monoclonal Antibodies and Cancer Therapy, Alan R. Liss, Inc., pp. 77-96 (1985)). Techniques developed for the production of “chimeric antibodies” by splicing the genes from a mouse antibody molecule of appropriate antigen specificity together with genes from a human antibody molecule can be used (e.g., Morrison, et al, Proc. Natl Acad. Sci. USA, 81:6851-6855 (1984); Neuberger, et al., Nature, 312:604-608 (1984); Takeda, et al., Nature, 314:452-454 (1985). Such chimeric antibodies are particularly useful for in vivo administration into human patients to reduce the development of host anti-mouse response. In addition, techniques described for the production of single chain antibodies (U.S. Pat. No. 4,946,778, which is incorporated herein by reference) can also be adapted.

Such antibody conjugates may be administered to a human patient prior to or simultaneously with donor cell engraftment. It is preferred that these conjugates are administered intravenously. Although the effective dosage for each antibody must be titrated individually, most antibodies may be used in the dose range of 0.1 mg/kg-20 mg/kg body weight. In cases where sub-lethal doses of irradiation are used, total body irradiation (TLI ) of a human recipient may be administered up to 7.5 Gy as a single dose or a combined total of 22 Gy administered in fractionated doses. Alternatively, TBI may be administered up to about 5.5 Gy.

The use of antisense strategies presents a theoretically simple tool to identify, with exquisite precision, the molecular mechanisms responsible for various cellular processes. It is based on the fact that each protein synthesized by a cell is encoded by a specific messenger mRNA (mRNA). If translation of a specific RNA is inhibited, the protein product derived from this translation will likewise be reduced. Oligonucleotide sequences, can therefore be designed to be complementary (antisense) to a specific target mRNA sequence, such as the β-chain and/or the δ-chain of TCR, and because of this complementarity, it will bind to the target sequence thereby inhibiting translation of that specific mRNA. An antisense oligonucleotide complementary to a particular mRNA is referred to herein as being “directed against” the product of translation of that message. It is believed that an antisense oligonucleotide, by hybridizing to the RNA and forming a complex, blocks target mRNA ribosomal binding causing translational inhibition. Alternatively, the duplex that is formed by the sense and antisense molecules may be easier to degrade. Other theories describe complexes that antisense RNA could form with complementary DNA to inhibit mRNA transcription. Thus, an antisense oligonucleotide might inhibit the translation of a given gene product by either directly inhibiting translation or through inhibition of transcription.

The following non-limited examples provide methods for identifying the cells that play a critical role in the resistance to engraftment of allogeneic bone marrow. All scientific and technical terms have the meanings as understood by one with ordinary skill in the art. The methods may be adapted to variation in order to produce compositions or devices embraced by this invention but not specifically disclosed. Further variations of the methods to produce the same compositions in somewhat different fashion will be evident to one skilled in the art.

Example I below demonstrates the utility of the present invention by clearly exemplifying the underlying discovery that αβ-TCR cells and γδ-TCR cells play a critical role in the resistance to engraftment of allogeneic bone marrow. Example 2 demonstrates that antibodies of the present invention, specific to αβ-TCR cells and γδ-TCR cells serve as useful tools in depleting αβ-TCR cells and γδ-TCR cells and thus increasing the induction of mixed allogeneic chimerism resulting in donor-specific tolerance with minimum morbidity. Alternate methods contemplated and understood by those skilled in the art include the use of that antisense DNA targeting of the genes that produce αβ-TCR cells and γδ-TCR cells or an alternate embodiment would utilize TBI for non-specific elimination of αβ-TCR cells and γδ-TCR cells.

### EXAMPLE I

### Identification of αβ-TCR Cells and γδ-TCR Cells That Play a Critical Role in the Resistance to Engraftment of Allogeneic Bone Marrow Using KO Mice

Mice

C57BL/6-Tcrb^{tm1Mom} (TCR-β KO (knock-out mouse), genes for production of TCR β-chain disrupted and deficiency in producing functional αβ-TCR cells); C57BL/6-Tcrd^{tm1Mom} (TCRδ-KO, genes for production of δ-chain disrupted and deficiency in producing functional γδ-TCR cells); C57BL/6-Tcrb^{tm1Mom} Tcrd^{tm1Mom} (TCR-β/δ double KO, genes for production of β-chain and δ-chain disrupted and deficiency in producing functional αβ-TCR and γδ-TCR cells); C57BL/6 -Cd8^{Tm/mak} (CD8-KO, genes for production of CD8 disrupted); C57BL/10-Cd4^{tm1} (CD4-KO, genes for production of CD4 disrupted); C57BL/6J-CD4^{tm/mak} (CD4-KO) and C57BL/6J (B6; H-2^{b}) recipient mice as well as B10.BR/SgSnJ (B10.BR; H-2^{k}) donor mice were purchased from the Jackson Laboratory (Bar Harbor, Me.). Knock-out mice were housed in a special pathogen-free barrier facility, while B6 and B10.BR mice were housed in the barrier facility at the Institute for Cellular Therapeutics. Mice were cared for according to National Institutes of Health animal care guidelines.

Chimera Preparation

Bone marrow was prepared from B10.BR donor mice (H2^{k}) as previously described. Briefly, BlO.BR donor mice were euthanized and tibias and femurs were harvested. Bone marrow was expelled from the bones with Media 199 (Gibco, Grand Island, N.Y.) containing 10 μg/ml Gentamicin (Gibco). The medium will be referred hereafter as MEM. The cells were filtered through sterile nylon mesh with 100 μm pores, centrifuged at 1000 rpm for 10 minute at 4° C., and resuspended in MEM. A cell count was performed and the cells were diluted to a final concentration of 15×10⁶ bone marrow cells per 1 ml of MEM.

Recipient mice were treated with 0 to 300 cGy total body irradiation from a cesium source (Gamma-cell 40, Nordion, Ontario, Canada). Animals were transplanted with 1 ml MEM containing 15×10⁶ B10.BR bone marrow cells via lateral tail vein injection within 4 hours of irradiation. All animals in groups treated with cyclophosphamide received a single intraperitoneal injection of 200 mg/kg cyclophosphamide (Sigma, St. Louis, Mo.) 48 hours after BMT.

Flow Cytometric Analysis

The level of chimerism was assessed 4 weeks after BMT by flow cytometric analysis of peripheral blood lymphocytes (PBL) using mAb against MHC antigens of donor and host origin. Fifty μl of whole blood obtained by tail bleeding of the mice were incubated at room temperature for 8 minutes with lysing buffer (8.29 g of NH₄ C1, 1.0 g of KHCO₃, and 0.0372 g Na₂EDTA in 1 liter H₂O; prepared in our laboratory) to eliminate red blood cells. The leukocytes were then incubated with 10 μl diluted mAb (details below) for 30 minutes at 4° C. in the dark. The appropriate dilution for the use of the mAb was determined in titration experiments prior to use. The cells were washed twice with 2 ml of FACS medium (0.36 g NaHCO₃, 1.0 g NaN₃ and 1.0 g bovine serum albumin in 1 liter Hanks Balanced Salt Solution; prepared in our laboratory) and centrifuged at 1000 rpm for 10 minutes at 4° C. Finally the cells were fixed with 1% paraformaldehyde in phosphate buffered saline (prepared in our laboratory). The analysis was carried out on a FACS-Calibur (Becton Dickinson Mountain View, Calif.) with CellQuest software (Becton Dickinson).

Monoclonal Antibodies

On day 28 after BMT antibodies specific for MHC Class 1 antigens of donor (FITC conjugated anti-H2K^{k}, 36-7-5, mouse IgG1) and recipient (PE conjugated anti-H2K^{b} AF6-88.5, mouse IgG2a) origin were used to determine the percentage of donor cell chimerism in the recipient's peripheral blood. Multi-lineage engraftment was assessed by staining with biotinylated anti-H2K^{k} (36-7-5, mouse IgG1), FITC conjugated anti-H2K^{b} (AF6-88.5, mouse IgG2a) and PE conjugated lineage markers 3 months after BMT. The biotinylated antibody was counter-stained with Streptavidin-Allophycocyanin (SA-APC). The following antibodies were used as lineage markers: anti-GR-1 (RB6-8C5, rat IgG2b); anti-MAC-1 (M1/70, rat IgG2b); anti-CD4 (RM4-5, rat IgG2a); anti-CD8α (53-6.7, rat IgG2a); anti-B220 (RA3-6B2, rat IgG2a); anti-NK1.1 (PK136, mouse IgG2a); anti-TCR-β chain (H57-597, hamster IgG); and anti-γδ-TCR (GL3, hamster IgG). The NK subpopulations were assessed by 4 color staining in naive B6, TCR-β KO, TCR-δ KO, TCR-β/δ KO and CD8 KO mice with FITC conjugated anti-TCR-β/γδ, Per CP conjugated anti-CD3e (145-2C11, hamster 1gG), APC conjugated anti-CD8α and PE conjugated NK subpopulation markers. The following antibodies were used as NK subpopulation markers: anti-NK1.1, anti-5E6 (5E6, mouse IgG2a), anti-2B4 (2B4, mouse IgG2b) and anti-DX5 (DX5, rat IgM). Non-specific background staining was controlled by using isotype control antibodies directed against irrelevant antigens conjugated with the same color as the experimental antibody (i.e. anti-TNP mouse IgG2a antigens and conjugated with PE served as isotype control for PE conjugated anti-H2K^{b} mouse IgG2a). All mAb were obtained from PharMingen (San Diego, Calif.). SA-APC was purchased from Becton Dickinson (Mountain View, Calif.).

Assessment of Graft-versus-Host Disease (GVHD)

The primary diagnosis of GVHD was based on previously described clinical criteria, which consist of diffuse erythema (particularly of the ear), hyperkeratosis of the foot pads, hair loss, weight loss, unkempt appearance, or diarrhea, (Bechomer, W. E., et al., Clin. Immunol. Immunopathol., 22:203-224 (1982)). At the time of sacrifice, sections of skin, tongue, liver, and small intestine were fixed in 10% buffered formalin, stained with hematoxylin and eosin, and processed for light microscopy.

Skin Grafting

Skin grafting was performed by a modification of the method of Billingham (Billingham, R. E., et al., The Wistar Institute Press, pp 1-26 (1961). Full-thickness tail skin grafts were harvested from the tails of B 1 0.BR and NOD mice. Recipient mice were anesthetized with Nembutal (pentobarbital sodium injection, Abbott, North Chicago, Ill.), and full-thickness graft beds were prepared surgically in the lateral thoracic wall preserving the panniculus carnosum. The grafts were covered with a double layer of Vaseline gauze and a plaster cast. Casts were removed on the seventh day and grafts were scored by daily inspection for the first month and then weekly thereafter for percentage of rejection. Rejection was defined as complete when no residual viable graft could be detected.

Statistical Analysis

Statistical significance was determined with a Student's one-way t test. The difference between groups was considered to be significant if P<0.05.

Chemotherapeutic Agents

Cyclophosphamide suppresses cell-mediated immunity and induces quantitative and qualitative changes in the lymphocyte repertoire (Hunninghake, G. W., et al., Immunology, 31:139-144 (1976)). The administration of cyclophosphamide results in leukopenia by depletion of mononuclear cell populations. At the same time cyclophosphamide can mediate a marked decrease in the cellular cytotoxic function of the remaining cells (Hunninghake, G. W., et al, Immunology, 31:139-144 (1976)). The administration of cyclophosphamide in the preparative regiment enhances allogeneic engraftment if it is administered 2 days after low dose TBI and bone marrow infusion. A similar effect does not occur if the cyclophosphamide is administered prior to marrow infusion in this model. It was hypothesized that the mechanism for this effect involves elimination of alloreactive T-cells from the recipient during the early stages of priming. In the present studies, the fact that mice lacking αβ- and γδ-TCR T-cells engraft with TBI alone suggests that these cell types are two major targets removed by cyclophosphamide in wild type recipients.

The level of engraftment in TCR- β/δ double KO mice was higher in animals that are conditioned with 300 cGy TBI alone as compared to animals conditioned with 300 cGy TBI and cyclophosphamide. It is hypothesized that this is due to the fact that proliferation of donor reactive T-cell clones is triggered by two days after BMT, rendering these cells an optimal target for cyclophosphamide. At the same time recipient-reactive T-cell clones of donor origin will proliferate against donor alloantigens as well. These cells will be depleted by the administration of cyclophosphamide along with the donor-reactive T-cell clones. Thus, the level of engraftment is higher in TCR-β/δ double KO mice when cyclophosphamide is not administered. Recipient-reactive donor T-cells will not be depleted in animals conditioned without cyclophosphamide. This will increase the level of donor chimerism, since T cells enhance engraftment through a graft vs. host reactivity. At the same time these cells could also mediate GVHD (Korngold, R., et al., Transplantation, 44:335-339 (1987)). The high mortality within 3 months in the group of TCR-β/δ double-KO mice transplanted without cyclophosphamide could be explained by the proliferation of host reactive donor T-cells mediating GVHD, and is supported by the histological evidence for GVHD in this cohort. An alternative hypothesis would suggest that cyclophosphamide is required for durable engraftment and that the high mortality in this group is a result of cessation of the production of self-renewing hematopoietic cells after engraftment of committed progenitors.

TBI does not completely eliminate donor-reactive T-cells from the recipient microenvironment, even at high doses. Davenport, et al., described that CD8⁺ T-cells of host origin are left behind in filly ablated mice (950 cGy). These cells are able to reject MHC mismatched donor bone marrow cells (Davenport, C., et al., The Journal of Immunology, 155:3742-3749 (1995)). The importance of this phenomenon has been shown clinically, when donor-reactive T-cell clones that were present before conditioning re-emerged after BMT and resulted in graft rejection (Voogt, P. J., et al., Lancet, 335:113-134 (1990)). In our studies we observed that cyclophosphamide is required to prevent rejection of allogeneic bone marrow grafts in mice conditioned with 300 cGy TBI, unless the animals lacked both αβ- and γδ-TCR⁺ T-cells. These data therefore strongly suggest that donor-reactive T-cells in the recipient hematopoietic environment are not completely removed by this dose of irradiation.

In syngeneic mice recipients, a low dose of TBI is required for conditioning if physiologic number of bone marrow cells is administrated (Down, J. D., et al., Blood, 77:661-669 (1991)). In CD8-KO mice treated with cyclophosphamide on day +2 after BMT, the kinetics of engraftment are similar to that observed in the syngeneic transplants. It is important to note that CD8 KO mice engraft without TBI, and at a very low irradiation dose, but do not engraft if cyclophosphamide is omitted from the conditioning. The level of engraftment is proportional to the irradiation dose and in this way resembles the characteristics of syngeneic engraftment. This data therefore confirm that host CD8⁺ cells as well as αβ-T cells and γδ-T cells each play a mechanistically different role in engraftment of MHC-disparate marrow.

This data suggests that more than one cell type mediate the rejection of fully MHC and minor antigen different bone marrow grafts. The data demonstrate a critical role for recipient αβ-TCR⁺ and γδ-TCR⁺ T-cells, but also CD8⁺ cells in the resistance to allogeneic bone marrow grafts. It is likely that the different cell types mediate rejection by different mechanisms. Interestingly, CD8⁺ cells were able to reject allogeneic bone marrow even in the complete absence of CD4⁺ cells, suggesting a CD4 independent mechanism. Targeting αβ- and γδ-TCR⁺ T-cells, as well as CD8⁺ T-cells in the recipient may allow a specific approach to the development of cell specific conditioning strategies to establish mixed chimerism with less toxicity. If chimerism could be achieved with minimal morbidity, and optimally if radiation could be eliminated completely, mixed chimerism could be more readily applied for tolerance induction, in gene therapy and treatment of non-malignant diseases such as autoimmune diseases and hematological disorders such as sickle cell disease and thalassemia.

### Mice Lacking Production of αβ-TCR⁺ plus γδ-TCR⁺ (TCR-β/δ KO) T-cells Engraft at a Significantly Lower TBI Dose

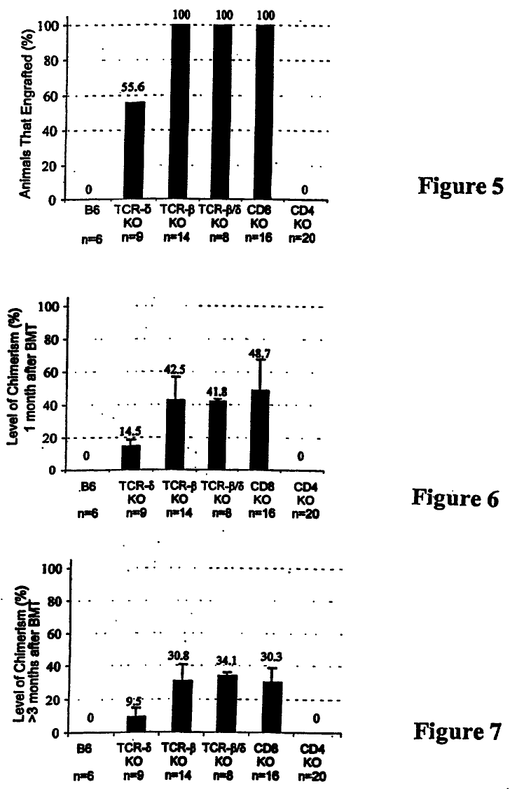

It has been previously demonstrated that normal mice require conditioning with 500 cGy TBI plus a single dose of cyclophosphamide on day +2 to engraft when transplanted with MHC and minor antigen disparate marrow (Colson, Y. L., et al., Journal of Immunology, 155:4179-4188 (1995)). To evaluate the role of host T-cells in engraftment of MHC and minor antigen disparate donor marrow, B6 (H-2^{b}) mice deficient in production of αβ-TCR⁺ γδ-TCR⁺ T-cells (TCR-β/δ double-KO) both αβ-TCR⁺ γδ-TCR⁺, CD8⁺, and CD4⁺ cells were utilized as recipients of allogeneic bone marrow grafts. All are H-2^{b} in MHC (B6). Engraftment occurred in 100% of TCR-β/δ double-KO mice conditioned with 300 cGy TBI and a single dose of cyclophosphamide two days after transplantation with 15×10⁶ bone marrow cells from B10.BR donors (n=8; FIG. 5). The level of chimerism was 41.8%±1.2% 1 month after BMT (FIG. 6). Similarly, 100% of TCR- β KO mice engrafted after conditioning with 300 cGY TBI plus CyP (n=14; FIG. 5). The level of chimerism was similar to that for the TCR- β/δ KO recipients (42.5%±14%; FIG. 6). The engraftment was durable and multilineage. Similarly, engraftment occurred in 100% of CD8 KO mice conditioned with 300 cGy TBI plus CyP (n=16; FIG. 5). The level of chimerism was 48.7%±18.1% at 1 month post-transplantation. At 3 months, donor chimerism was 30.3%±8.4% and remained stable for a period of time greater than six months, as shown if FIG. 7. In striking contrast, only 5 of 9 (56%) γδ-TCR KO recipients engrafted, and the level of chimerism was significantly lower in mice that engrafted (14.5%±4.3%) compared to the TCR-β KO (P<0.005) and TCR β/δ double KO mice (P<0.005), as shown FIG. 6. CD4 KO mice conditioned in a similar fashion did not engraft (n=20), suggesting that host CD8⁺ cells and αβ- and γδ-TCR⁺ T dells play a major role in alloresistance to engraftment while CD4⁺ cells do not. Moreover, the effector cells in alloresistance did not require CD4⁺ cells, since CD4 KO mice require significant levels of conditioning for engraftment. As expected, B6 control mice did not engraft when conditioned and transplanted in similar fashion (n=6, FIG. 5).

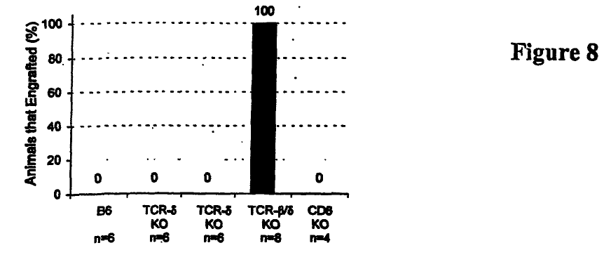

It is hypothesized that CyP on day +2 relative to the marrow infusion removes alloreactive T-lymphocytes that have been activated by alloantigen. To evaluated the contribution of CyP to the conditioning approach and further define which host cells were the target on day +2 CyP infusion, TCR-β KO, TCR-δ KO, TCR-β/δ KO, and CD8 KO mice were conditioned with 300 cGy of TBI and transplanted with 15×10⁶ B10.BR bone marrow cells, FIG. 8. Neither TCR-β KO (n=6), the TCR-δKO (n=6), nor CD8 KO (n=4) mice engrafted. One hundred percent of the TCR-β/δ KO mice engrafted (n=6; Table 1, shown below).

TABLE 1

| | | | Level of | Level of |
|---|---|---|---|---|
| | | Frequency of | Chimerism | Chimerism |
| | Number | Engraftment | (% ± SD) | (% ± SD) |
| Mouse Strain | of Mice | (%) | 1 month | 4 months |
| Controls | 6 | 0 | 0 | 0 |
| TCR-βKO | 6 | 0 | 0 | 0 |
| TCR-δKO | 6 | 0 | 0 | 0 |
| TCR-β/δKO | 6 | 100 | 77.7 ± 23.5 | 76.8 ± 40.2 |
| CD8 KO | 4 | 0 | 0 | 0 |
| This table shows the frequency of engraftment and level of chimerism in various KO-mice conditioned with 300 cGy. TBI alone and transplanted with 15 × 10⁶ allogeneic; bone marrow cells from B10.BR donors. Without the administration of cyclophosphamide, only TCR-β/δ double KO mice engrafted. Surprisingly the level of chimerism in these animals was higher than that for TCR-β/δ double KO mice | | | | |
| #conditioned in the same way, but who also received 200 mg/kg cyclophosphamide two days after transplantation. A 50% mortality was noted within 3 months of BMT in the TCR-β/δ KO group conditioned with radiation alone, while all TCR-β/δ double KO mice conditioned with 300 cGy plus cyclophosphamide survived. | | | | |

Interestingly, the level of engraftment at 1 month after BMT was approximately twice as high in TCR-β/δ double KO mice conditioned with TBI alone (77.7%±23.5%) (Table 1) as compared to animals conditioned with TBI and cyclophosphamide (41.8%±1.2%) (FIG. 6). A dose-titration of irradiation revealed that 100% of mice conditioned with 200 cGy or 100 cGy TBI engrafted (FIG. 1). In fact, 85.7% of TCR-β/δ double KO mice (n=7) engrafted without any TBI conditioning (FIG. 1). The level of chimerism was directly correlated with the degree of conditioning (FIGS. 2, 3 and 4 ). In this group of animals (FIGS. 1-4), the engraftment was durable as assessed by flow cytometric analysis monthly for up to 5 months. Therefore, αβ as well as γδ T-cells are the critical cyclophosphamide-sensitive cells in mediating resistance to alloengraftment.

Interestingly, the level of chimerism after BMT was approximately twice as high in TCR-β/δ KO mice conditioned with 300 cGy of TBI alone (76.1%±10.$%) versus those conditioned with 300 cGy of TBI plus CyP (42%±1.2%). In the absence of the CyP-sensitive αβ- and γδ-TCR⁺ T cells, when CyP is not required for engraftment to occur, one can evaluate the impact of conditioning on the level of chimerism that results. Clearly, CyP itself somewhat impairs engraftment of the donor marrow. However, in the presence of host αβ- and γδ-TCR⁺ T cells, CyP is critical to overcoming the barrier for alloresistance since 0% of the TCR-β and TCR-δ single KO recipients engraft without CyP when conditioned with 300 cGy TBI.

### Multilineage Chimerism Occurs Following Partial Conditioning

The pluripotent hematopoietic stem cell produces at least 11 different cell lineages. To confirm that the engraftment in TCR-β/δ double KO recipients reflects engraftment of the pluripotent stem cell, animals were followed for more than 3 months. The engraftment achieved in TCR-β/δ double KO mice conditioned with 300 cGy irradiation and administration of cyclophosphamide on day +2 was durable (FIG. 1C) and multi-lineage. Three color flow cytometric analysis showed the presence of multiple myeloid and lymphoid lineages of donor and host origin in all engrafted animals. The T-cell lineages deficient in the knock-out animals could be found in the transplant recipients and were all of donor origin.

### Host αβ-T Cells and, to a Lesser Extent, γδ-T Cells Influence Engraftment of Allogeneic Marrow

To examine the differential roles of host αβ- and γδ-TCR⁺ cells on engraftment, mice defective in production of αβ-TCR⁺ cells only were utilized as recipients. TCR-β KO mice were conditioned with 300 cGy TBI plus cyclophosphamide on day +2. Engraftment occurred in 100% of the animals (n=14, FIG. 1A). The level of engraftment (42.5%±14.0%) was similar to the level of engraftment in TCR-β/δ double KO mice (41.8%±1.2%) (FIG. 1B). The engraftment was also durable (FIG. 1C) and multi-lineage (data not show). However, when cyclophosphamide was omitted from the preparative regimen, no engraftment occurred in TCR-β KO mice (n=6, Table 1), supporting the hypothesis that recipient γδ-T cells are also critical effector cells in alloresistance to engraftment. These data also confirm that alloreactive T-cells are the primary target for cyclophosphamide conditioning.

When animals lacking only γδ-TCR⁺ cells were used as recipients, only 5 of 9 (55.6%) engrafted after conditioning with 300 cGy of TBI plus cyclophosphamide (FIG. 1A). None engrafted if cyclophosphamide was omitted (n=6, Table 1). The level of chimerism in TCR-δ KO mice that engrafted was 14.5%±4.3%, significantly lower than in TCR-β KO (P<0.005) or TCR-δ double-KO mice (P<0.005) (FIG. 1B). The engraftment achieved was durable (FIG. 1C). These data confirm that host αβ- as well as γδ-TCR⁺ cells exert a critical and independent influence on engraftment of allogeneic marrow, although αβ-TCR⁺ cells seem to be more important since mice deficient in production of only γδ-T cells showed a lower percentage of engraftment for a given dose of conditioning and a lower level of overall chimerism.

### Host CD8⁺ Cells Influence Engraftment While CD4⁺ Cells Do Not: Evidence for a CD4⁻ Independent Mechanism for Alloresistance

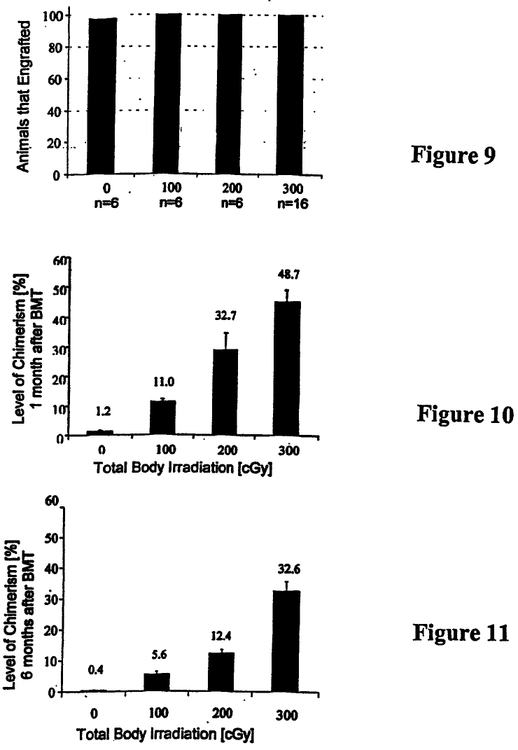

The role of CD8⁺ and CD4⁺ cells in the resistance to allogeneic bone marrow engraftment was examined using CD8-KO and CD4-KO mice. As shown in FIG. 9, engraftment occurred in 100% of mice lacking CD8⁺ cells conditioned with 300 cGy irradiation and cyclophosphamide on day +2 (n=16, Table 2, shown below).

TABLE 2

| | | | | Level of donor |
|---|---|---|---|---|
| | | | | chimerism |
| | Gene | | | (% ± SD) |
| | Knocked | Number | Animals that | 1 month after |
| Mouse strain | Out | of mice | engrafted [%] | BMT |
| C57BL/6J | — | 6 | 0 | 0 |
| C57BL/10- | CD4 | 5 | 0 | 0 |
| Cd4^{tml} | | | | |
| C57BL/6- | CD4 | 15 | 0 | 0 |
| Cd4^{tmlmak} | | | | |
| C57BL/6 | CD8 | 16 | 100 | 48.7 ± 18.1 |
| Cd8alpha^{tmlmak} | | | | |

CD4 or CD8 KO mice were conditioned with 300 cGy TBI and a single dose of 200 mg/kg of cyclophosphamide IP two days after BMT. The level of chimerism was analyzed in peripheral blood 1 month after BMT by flow cytometry. The level of donor chimerism is shown for the animals that engrafted. CD4-KO mice (C57BL/10Cd4^{tm1}) share the same MHC as B6 and CD8-KO mice, but are disparate in the non-MHC minor antigens. CD4-KO mice (C57BL/10Cd4^{tm/mak}) are congeneic at all loci with B6.

The level of chimerism was 48.7%±18.1% 1 month after transplantation. At 3 months after transplantation, the level of chimerism was 30.3%±8.4%, and remained stable thereafter with 32.6%±9.5% when analyzed monthly for ≧6 months after BMT (Table 3, shown below).

TABLE 3

| Time after BMT (months) | Donor-Specific Cells (% ± SD) |
|---|---|
| 1 | 48.7 ± 18.1 |
| 3 | 30.3 ± 8.4 |
| 6 | 32.6 ± 9.5 |

The level of donor cell chimerism in peripheral blood after transplantation of 15×10⁶ untreated B10.BR bone marrow cells was followed by flow cytometric analysis for up to 6 months. Although the level of chimerism decreased slightly at 3 months compared to 1 month, none of the animals lost their chimerism and the level remained stable thereafter.

In striking contrast, none of the mice lacking CD4⁺ cells engrafted when transplanted with the same dose of bone marrow following similar conditioning (Table 2). Initially CD4-KO mice that shared the same MHC as B6 and CD8-KO mice, but disparate in the non-MHC minor antigens, were used (n=5). To exclude the possibility that these minor antigenic differences could influence engraftment, the experiments were repeated using another strain of CD4-KO mice congeneic at all other loci with B6 mice as recipients (n=15). Again no engraftment was occurred in any of these animals (Table 2). These data suggest a critical role for CD8⁺ cells in alloresistance to engraftment. Surprisingly, CD8 effect does not require help—most T cell activation does require help.

### Engraftment in CD8-KO Mice Can Be Achieved Without Irradiation, But Not Without Cyclophosphamide

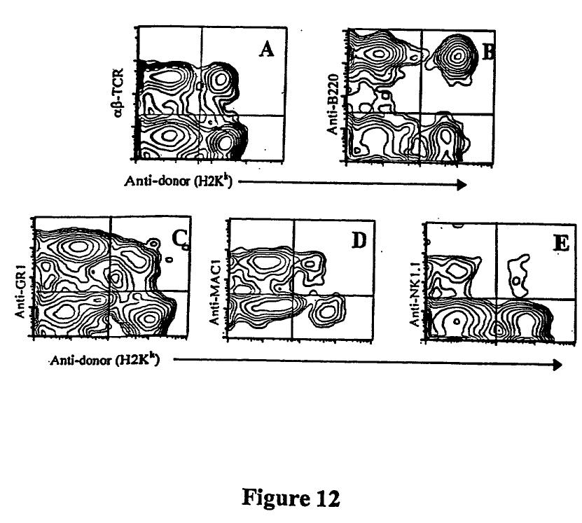

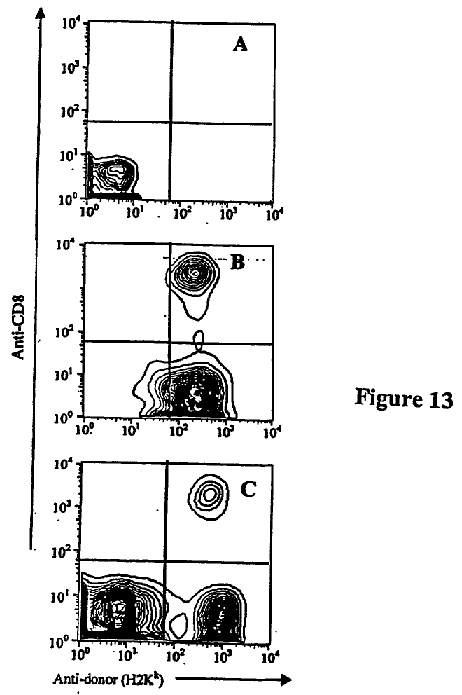

When CD8-KO mice were conditioned with 300 (n=16); 200 (n=6); 100 (n=6); or 0 (n=6) cGy TBI, transplanted with 15×10⁶bone marrow cells and injected with 200 mg/kg cyclophosphamide (i.p.) on day +2, 100% of the animals engrafted. The level of chimerism was proportional to the dose of TBI (FIG. 10). Engraftment was durable in all animals conditioned with 300, 200, or 100 cGy of TBI for a minimum follow of 4 months (FIG. 11). The engraftment was also multilineage (FIG. 12) and the CD8 lineage was of only donor origin (FIG. 13). Half of the animals conditioned with cyclophosphamide but not TBI lost their chimerism completely within 4 months after BMT. The level of engraftment in the other animals in that group was at the threshold of detectability (FIG. 3B). In striking contrast, when CD8-KO recipients (n=4) were conditioned with 300 cGy TBI alone and transplanted with B10.BR marrow, engraftment did not occur (Table 1), suggesting that the cells present in CD8 KO mice responsible for allorejection are very sensitive to cyclophosphamide but much less sensitive to radiation.

### Development of GVHD

TCR β/δ KO mice (in FIG. 10) irradiated with 0 to 300 cGy TBI and reconstituted with 15×10⁶ B10.BR bone marrow cells were followed for ≧5 months. Clinical evidence for GVHD, such as diffuse erythema, dermalitis, hyperkeratosis of the footpads, diarrhea, or body weight loss, was observed in the majority of recipients. The severe diffuse erythema and dermatitis could cause the deformation and even loss of the ears. These mice died or had to be euthanized due to extensive weight loss and severe skin lesions. GVHD was detected histologically in all the tissues of skin, tongue, intestine and liver from 3 representative mice.

### Evidence for Specific Tolerance In Vivo to Donor-Type Skin Grafts

Skin grafting was performed to assess donor-specific tolerance in vitro. Five unmanipulated TCR-β/δ KO mice (H2^{b}) received full-thickness skin grafts of both B10/BR (H2^{k}) and NOD (H2^{d}) origin. All grafts survived more than 160 days, demonstrating that naive TCR-β/δ KO mice do not reject skin allografts. Both donor-specific (B10.BR) and MHC-disparate third-party (NOD) skin grafts were placed on the chimeric TCR-β/δ KO mice with different levels of donor chimerism (see Table 4 below).

TABLE 4
Donor-specific tolerance in mixed allogeneic chimeras^{a}

| | | | % Donor | Survival time of skin graft | |
|---|---|---|---|---|---|
| | | | Chimerism | (days) | |
| Animal | N | TBI | (4 months) | NOD | B10.BR |
| Control | 5 | None | None | >160 | >160 |
| A | 1 | 0 | 0 | 82^{b} | 82^{b} |
| B | 1 | 0 | 2.5 | 12 | >160 |
| C | 1 | 200 | 71.3 | 8 | >160 |
| D | 1 | 300 | 21.0 | 9 | >160 |

^{a}TCR-β/δ KO mice (H2^{b}) were treated with different doses of TBI and infused with 15 × 10⁶ B10.BR (H2^{k}) bone marrow. Each chimeric animal received skin grafts from donor-specific (B10.BR) and third-party (NOD, H2^{d}) strains 4 to 5 months after BMT. Five naïve TCR-β/δ KO mice were used as controls.
^{b}Animal was found dead on 82 days post skin transplantation.

Grafts were assessed daily for the first 4 weeks and weekly thereafter for evidence of rejection. The single non-chimeric mouse accepted the donor-specific and third party skin grafts in a fashion similar to that observed in naive TCR-β/δ KO mice. In all other recipients, donor-specific allogeneic skin grafts were accepted by the mice with chimerism (range from 2.5% to 71.3%), while third-party skin grafts were promptly rejected. These data therefore demonstrate that TCR-β/δ KO recipients that engraft as chimeras exhibit donor-specific tolerance but are immunocompetent to reject MHC-disparate third-party allografts.

### Marrow from TCR-β KO, TCR-β KO, TCR-β/δ KO and CD8 KO Mice Contains NK Cells

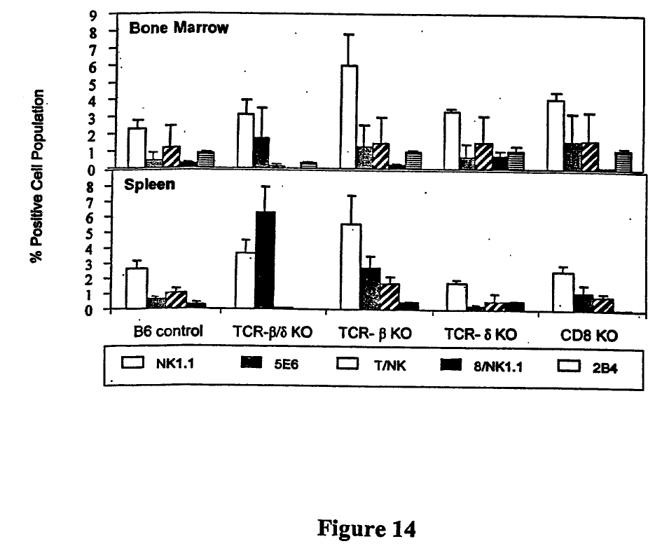

T cells as well as NK cells have been implicated in alloresistance to engraftment. A number of NK subfamilies have been described, including 5E6, 2B4, T/NK cells, and CD8⁺ NK cells. Marrow and splenocytes from CD8, TCR-β/δ, TCR-β, and TCR-δ KO mice were analyzed by four color flow cytometry to enumerate which NK subfamilies might be absent (FIG. 13). All KO mice produced NK1.1⁺ and 5E6⁺ cells in marrow and spleen at levels similar to wild type B6 controls. Marrow from the TCR-β/δ KO mice contained a significantly lower number of T/NK cells than B6 (P<0.0019). 2B4⁺ NK cells were also significantly reduced (P=0.0005) and CD8⁺/NK cells were virtually absent (P=0.0046). CD8 KO mice lack CD8⁺/NK cells (FIG. 14) as well as CD8⁺ T cells, as expected (data not shown). One could therefore hypothesize that the T/NK subfamily present in CD8 KO mice but lacking in TCR-β/δ KO mice may represent the CyP-sensitive cell and may explain why CyP is not required to achieved engraftment in TCR-β/δ KO mice.

In the present studies we also observed a critical role for host CD8⁺ cells in regulating engraftment. Durable, multi-lineage engraftment occurred in all CD8-KO mice conditioned with any dose of TBI as long as cyclophosphamide was administered. The level of chimerism was directly correlated with the dose of TBI. In striking contrast, none of the CD4-KO mice conditioned with as high as 300 cGy TBI plus cyclophosphamide engrafted when transplanted in a similar fashion. These results demonstrate that CD8⁺ cells in the recipient hematopoietic microenvironment play a critical role in marrow rejection. The requirement for cyclophosphamide on day +2 suggests that conventional T-cells rather than NK cells are the primary effector cells since NK cells do not require priming, while T-cells do.

NK cells have been implicated to play a major role in marrow rejection. Several subfamilies of NK cell have been described, including such as 5E6 (Ly49C+I), 2B4 and DX5. 5E6⁺ NK cells comprise 50% of NK cells and have been demonstrated to influence engraftment and hematopoiesis (Sentman, C. L., et al., Eur. J. Immunol., 21:2821-2828 (1991); and Semman, C. L., et al., J. Exp. Med., 170:191-202 (1998)). We observed that the NK1.1⁺ and Ly49C+1 (5E6) NK subsets are present in TCR-β KO, TCR-β KO, TCR-β/δ and CD8 KO mice at levels similar to that for B6 control mice. The fact that mice which lack αβ and/or γδ T cells engraft with less conditioning strongly supports a critical role for conventional T-cells rather than NK cells in alloresistance. Moreover, the fact that TCR-β/δ KO mice have no NK/T cells makes it likely that T/NK cells contribute also to alloresistance to engraftment but that conventional T cells are the primary effector cell. Thus, “conventional” NK cells are not as important as believed by those in the art, but T/NK cells are.

The classic pathway to initiate cytotoxicity mediated by CD8⁺ T-cells involves the help of CD4⁺ cell (Cantor, H., et al., J. Exp. Med., 141:1376-1389 (1975)). However, pathways of CD4⁺ cell-independent initiation of cytotoxicity have been described. Purified CD8⁺ cells can mount cytolytic responses without CD4 mediated help in vitro (Singer, A., et al., J. Immunol., 132: 2199-2209, (1984); and (Sprent, J., et al., J. Exp. Med., 163:998-1011 (1986)) and in vivo (Sprent, J., et al., J. Exp. Med., 163:998-1011 (1986). Another CD4-independent CD8-mediated mechanism of cytotoxicity is an NK-like mechanism of alloreactivity (Davenport, C., et al., Journal of Immunology, 154:2568-2577 (1995)). A number of groups have described an overlap between T-cells and NK-cells. Dennert, et al., have suggested that CD3⁺ NK 1.1⁺ cells can develop into CD8⁺ cytotoxic T-cells during acute rejection of allogeneic bone marrow grafts (Dennert, G., et al., Immunogenetics, 31:161-168 (1990)). While T-cell-mediated cytotoxicity usually requires activation and takes about 7-8 days to generate a cytotoxic response, rejection via NK-cells occurs within 4-5 days (Murphy, W. J., et al., Journal of Experimental Medicine, 166:1499-1509 (1987)). However, the early events of alloreactivity for T-cell activation take only hours after exposure to antigen (Cebrian, M., et al., J. Exp. Med., 168:1621-1637 (1987); and Testi, R., et al., J. Immunol., 142:1854-1860 (1989)).

The data of the present invention demonstrate a critical role for a CD4-independent CD8-mediated mechanism that mediates resistance to engraftment in recipients of allogeneic bone marrow. Although this could be due to T-cells or T/NK cells, the fact that αβ-TCR⁺ T-cells play a significant role in alloresistance to engraftment and that TCR-β/δ KO mice produce NK cells strongly supports a critical role for conventional T-cells.

### EXAMPLE II

### Use of Anti-αβTCR and/or Anti-γδTCR Monoclonal Antibodies for Conditioning a BMT Recipient

Hematopoietic stem cell (HSC) chimerism induces tolerance for solid organ allografts. The clinical application of this technique is limited by the morbidity and mortality of fully ablative conditioning. We previously reported that conditioning of the recipient with anti-lymphocyte globulin (ALG) (day −3); 300 cGy TBI (day 0) followed by a single dose of cyclophosphamide (CyP) (day +2) resulted in durable chimerism in MHC plus minor antigen disparate mice. In the present study, monoclonal antibodies (mAb) directed against αβ or γδ T-cells were administrated to mice to define which cells in the recipient must be depleted for allogeneic engraftment to result. B10 recipients (H2K^{b}) were pretreated i.v. with 100 mg of anti-αβTCR alone, anti-γδTCR alone and both of mAb on day −3. On day 0, recipients were conditioned with 0, 100, 200 or 300 cGy and transplanted with 15×10⁶ B10.BR (H2K^{k}) bone marrow cells followed by 200 mg/kg i.p. CyP on day +2. With anti-γδTCR pretreatment and 300 cGy/CyP, only 33.3% (n=6) of animals engrafted. In striking contrast, 100% of recipients pretreated with anti-αβTCR alone or anti-αβ+γδTCR engrafted with 100, 200, and 300 cGy TBI. Of those recipients receiving no TBI, 85.7% engrafted when treated with only anti-αβTCR while 100% engrafted after anti-αβ and anti-γδTCR treatment. The level of chimerism directly correlated with the degree of TBI conditioning and was similar between the two groups.

### Materials and Methods

Determination of Chimeras

The engraftment was assessed by flow cytometric analysis of peripheral blood lymphocytes using monoclonal antibodies (mAb) against MHC antigens of donor and host origin.

The level of chimerism was determined by the percentage of donor lymphocytes.

Skin Grafting

A donor-specific B10.BR graft and a third-party BALB/c (H2^{d}) graft were transplanted on each side of lateral thoracic wall per animal at the same time. Rejection was defined as complete when no residual viable graft could be detected.

### Results

With anti-γδTCR pretreatment and 300 cGy TBI/CyP, only 33.3% of animals engrafted. In striking contrast, 100% of recipients pretreated with anti-αβTCR alone or anti-αβ +anti-γδTCR engrafted in this model (Table 5).

TABLE 5
Allogeneic chimeras (B10.BR → B10)

| mAb | N | Engrafted |
|---|---|---|
| Anti-γδTCR | 6 | 33.3% |
| Anti-αβTCR | 16 | 100% |
| Anti-αβ/γδTCR | 8 | 100% |

With anti-αβTCR pretreated and CyP, 100% engraftment also achieved with as low as 100 and 200 cGy TBI. Of those recipients receiving no TBI, 90.9% mice engrafted at 30 days. Mixed allogeneic chimerism was stable in this group of mice when conditioned with ≧100 cGy TBI (Table 6 and Table 7)

TABLE 6
Allogeneic chimeras (B10.BR → B10) After 1 Month

| | | | % Donor |
|---|---|---|---|
| TBI (cGy) | N | Engrafted (%) | Chimerism |
| 0 | 11 | 90.9 | 0.43 ± 0.22 |
| 100 | 6 | 100 | 25.6 ± 4.5 |
| 200 | 11 | 100 | 66.9 ± 6.6 |
| 300 | 16 | 100 | 89.5 ± 3.6 |

TABLE 7
Allogeneic chimeras (B10.BR → B10)
% Donor Chimerism (mean ± SD)

| | | % Donor Chimerism (mean ± SD) | | | | |
|---|---|---|---|---|---|---|
| TBI (cGy) | N | 1 month | N | 2 months | N | 3 months |
| 0 | 11 | 0.43 ± 0.22 | 5 | 0 | 5 | 0 |
| 100 | 6 | 25.6 ± 4.5 | 5 | 28.1 ± 1.9 | 5 | 28.6 ± 1.0 |
| 200 | 11 | 66.9 ± 6.6 | 3 | 60.8 ± 6.2 | 2 | 54.6 ± 6.5 |
| 300 | 16 | 89.5 ± 3.6 | 2 | 84.4 ± 2.7 | 1 | 84.1 |

With both mAb of anti-αβ and anti-γδTCR pretreatment/CyP, 100% engraftment also achieved with 0, 100, 200 cGy TBI at 30 days. Mixed allogeneic chimerism was durable when conditioned with ≧100 cGy TBI (Table 8).

TABLE 8
Allogeneic chimeras (B10.BR → B10)

| | | | % Donor | | % Donor |
|---|---|---|---|---|---|
| | | Engrafted | Chimerism | Engrafted | chimerism |
| TBI (cGy) | N | 30 days | 30 days | 120 Days | 120 days |
| 0 | 6 | 100% | 0.64 ± 0.29 | 0 | 0 |
| 100 | 5 | 100% | 17.8 ± 5.0 | 100% | 9.8 ± 5.2 |
| 200 | 5 | 100% | 52.8 ± 7.4 | 100% | 25.3 ± 2.4 |
| 300 | 8 | 100% | 76.7 ± 5.6 | 100% | 54.3 ± 13.7 |

The level of chimerism directly correlated with the degree of TBI conditioning and was similar between the two groups pretreated with anti-αβTCR alone or anti-αβ+γδTCR.

The chimeras accepted donor-type skin grafts (>100 days), but promptly rejected MHC-disparate third party BALB/c (H2K^{d}) skin grafts (MST=12.3 days ±1.5), irrespective of level of donor chimerism.

### EXAMPLE III

### Use of Anti-αβTCR and Anti-CD8 Monoclonal Antibodies for Conditioning a BMT Recipient

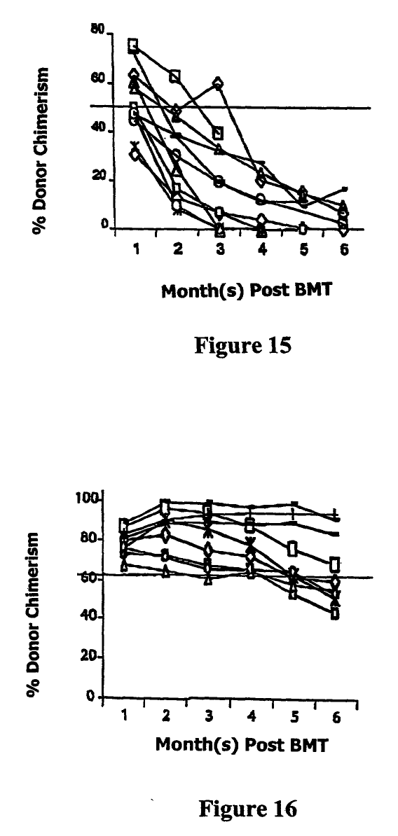

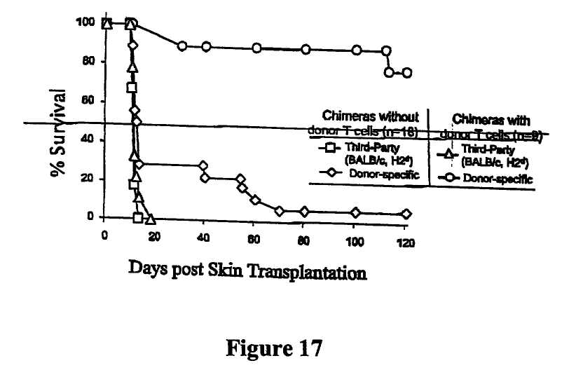

Hematopoietic stem cell (HSC) chimerism induces tolerance for solid organ grafts. In this Example, recipient B57BL/10 (H2^{b}) mice were pretreated in vivo with mAbs anti-αβ-TCR and anti-CD8 3 days before TBI (day 0) and transplanted with 15×10⁶ allogeneic (B 10.BR; H2^{k}) marrow cells. When recipients were pretreated with anti-αβ-TCR and anti-CD8 mAbs and conditioned with 0, 100, 200 or 300 cGy TBI, engraftment occurred in 0 (n=6), 20% (n=5), 75% (n=16) and 94% (n=16) mice one month post BMT, respectively. In those animals that engrafted from all groups, some animals exhibited multilineage production, including donor T cells, while others had only donor B cell, NK cell, macrophage and granulocyte production. Animals without donor T cell engraftment lost their chimerism gradually within 6 months (FIG. 15). Moreover, they rejected the donor and third-party skin grafts with a time course similar to naive controls even when they still had significant levels of donor chimerism. In animals with donor T cell production, mixed chimerism remained stable for ≧6 months (FIG. 16). Donor skin graft survival was prolonged in all animals in this group and the majority (7 out of 9) of the donor skin grafts were accepted permanently, while MHC-disparate third party grafts were rejected promptly (FIG. 17). These results indicate that pretreatment of the recipient with anti-αβ-TCR and anti-CD8 can reduce the TBI requirement for establishing mixed chimerism. However, donor-specific tolerance was observed only in mixed chimeras with donor T cell production, suggesting a critical role for T cells in maintenance of tolerance.

This model may provide a more acceptable clinical approach for the induction of donor-specific transplantation tolerance.

A wide variety of uses are encompassed by the invention described herein, including, but not limited to, the conditioning of recipients by non-lethal methods for bone marrow transplantation in the treatment of diseases such as hematologic malignancies, infectious diseases such as AIDS, autoimmunity, enzyme deficiency states, anemias, thalassemias, sickle cell disease, and solid organ and cellular transplantation.

The foregoing description is considered as illustrative only of the principles of the invention. The words “comprise,” “comprising,” “include,” “including,” and “includes” when used in this specification and in the following claims are intended to specify the presence of one or more stated features, integers, components, or steps, but they do not preclude the presence or addition of one or more other features, integers, components, steps, or groups thereof. Furthermore, since a number of modifications and changes will readily occur to those skilled in the art, it is not desired to limit the invention to the exact construction and process shown described above. Accordingly, all suitable modifications and equivalents may be resorted to falling within the scope of the invention as defined by the claims which follow.

The embodiments of the invention in which an exclusive property or privilege is claimed are defined as follows:

1. A method for conditioning a recipient for bone marrow transplantation comprising subjecting said recipient to a composition that specifically depletes αβ-, and γδ-TCR⁺ T cells and/or CD8⁺ T cells in the recipient hematopoietic microenvironment, followed by transplantation with a donor cell preparation containing hematopoietic stem cells from a donor that are matched at the major histocompatibility complex class I K locus with the recipient hematopoietic microenvironment.

2. The method of claim 1 in which said composition comprises antibodies specific for αβ-, and γδ-TCR⁺ T cells and/or CD8⁺ T cells.

3. The method of claim 1 in which said composition comprises antisense DNA that is directed against the precursors of αβ-, and γδ-TCR⁺ T cells and/or CD8⁺ T cells.

4. The method of claim 3 wherein antisense DNA alters the translation of the α-chain, β-chain, γ-chain, or δ-chain of TCR⁺ T cells.

5. The method of claim 3 wherein antisense DNA alters the transcription of the α-chain, β-chain, γ-chain, or δ-chain of TCR⁺ T cells.

6. The method of claim 1 in which said composition a cytotoxic drug specific for αβ-, and γδ-TCR⁺ T cells and/or CD8⁺ T cells.

7. The method of claim 1 wherein the recipient is further conditioned by subjecting the recipient to a total dose of total body irradiation of less than or equal to 300 cGy.

8. The method of claim 1 wherein the recipient is further conditioned by subjecting the recipient to an alkylating agent.

9. The method of claim 8 wherein said alkylating agent is cyclophosphamide.

10. The method of claim 1 wherein said composition specific to αβ-, and γδ-TCR⁺ T cells and/or CD8⁺ T cells in the recipient hematopoietic microenvironment totally eliminates said cells from the recipient hematopoietic microenvironment.

11. A method for conditioning a recipient for bone marrow transplantation comprising subjecting said recipient treatment with a total dose of total body irradiation from 100 to 300 cGy, and treating the patient with a composition that specifically depletes αβ-, and γδ-TCR⁺ T cells and/or CD8⁺ T cells in the recipient hematopoietic microenvironment, followed by transplantation with a donor cell preparation containing hematopoietic stem cells from a donor that are matched at the major histocompatibility complex class I K locus with the recipient hematopoietic microenvironment.

12. The method of claim 11 wherein the recipient is further treated with an alkylating agent before, during, or after exposure to said composition that specifically depletes αβ-, and γδ-TCR⁺ T cells and/or CD8⁺ T cells in the recipient hematopoietic microenvironment.

13. The method of claim 12 wherein said alkylating agent is cyclophosphamide.

14. A method of partially or completely reconstituting a mammal's lymphohematopoietic system comprising administering to the mammal a composition that specifically depletes αβ-, and γδ-TCR⁺ T cells and/or CD8⁺ T cells in the recipient hematopoietic microenvironment, followed by transplantation with a donor cell preparation containing hematopoietic stem cells from a donor that are matched at the major histocompatibility complex class I K locus with the recipient hematopoietic microenvironment.

15. The method of claim 14, in which the mammal suffers from autoimmunity.

16. The method of claim 15 in which the autoimmunity is diabetes.

17. The method of claim 15, in which the autoimmunity is multiple sclerosis.

18. The method of claim 15, in which the autoimmunity is sickle cell.

19. The method of claim 15, in which the autoimmunity is anemia.

20. The method of claim 15, in which the mammal suffers from a hematologic malignancy.

21. The method of claim 14, in which the mammal requires a solid organ or cellular transplant.

22. The method of claim 14, in which the mammal suffers from immunodeficiency.

23. A method for conditioning a recipient for bone marrow transplantation comprising subjecting said recipient treatment with a total dose of total body irradiation from 100 to 300 cGy, and treating the patient with a composition that specifically depletes αβ-TCR⁺ T cells and/or CD8⁺ T cells in the recipient hematopoietic microenvironment, followed by transplantation with a donor cell preparation containing hematopoietic stem cells from a donor that are matched at the major histocompatibility complex class I K locus with the recipient hematopoietic microenvironment.

24. A method for conditioning a recipient for bone marrow transplantation comprising subjecting said recipient to a composition that specifically depletes αβ-TCR⁺ T cells and CD8⁺ T cells in the recipient hematopoietic microenvironment, followed by transplantation with a donor cell preparation containing hematopoietic stem cells from a donor that are matched at the major histocompatibility complex class I K locus with the recipient hematopoietic microenvironment.
